(19) United States
(12) Patent Application Publication
(10) Pub. No.: US 2025/0144111 A1
(43) Pub. Date: May 8, 2025

(54) TAUROLIDINE TREATMENT FOR MYC-EXPRESSING TUMORS IN MAMMALIAN BODIES

(71) Applicant: CorMedix Inc., Berkeley Heights, NJ (US)

(72) Inventors: Bruce Reidenberg, Rye, NY (US); Robert DiLuccio, Viera, FL (US)

(21) Appl. No.: 18/795,424

(22) Filed: Aug. 6, 2024

Related U.S. Application Data
(63) Continuation of application No. 17/560,453, filed on Dec. 23, 2021, now Pat. No. 12,053,478.
Application No. 17/560,453 is a continuation of application No. 16/558,496, filed on Sep. 3, 2019, now abandoned.
Application No. 16/558,496 is a continuation-in-part of application No. 15/403,876, filed on Jan. 11, 2017, now abandoned.
(60) Provisional application No. 62/277,243, filed on Jan. 11, 2016.
(60) Provisional application No. 62/725,650, filed on Aug. 31, 2018.

(51) Int. Cl. A61K 31/549; A61K 9/16; A61K 31/047; A61K 31/132; A61K 45/06

(52) U.S. Cl. CPC A61K 31/549; A61K 9/167; A61K 31/047; A61K 31/132; A61K 45/06

(57) ABSTRACT

A method for treating a cancer which overexpresses any of N-myc genes, C-myc genes and/or L-myc genes in a mammalian body, the method comprising:
administering a composition to the mammalian body, wherein the composition comprises at least one from the group consisting of taurolidine; taurultam; taurinamide; methylene glycol; taurultam and taurinamide in a ratio of 1 taurultam:7 taurinamide; and taurultam, taurinamide and methylene glycol in a ratio of 1 taurultam:7 taurinamide:1 methylene glycol.

(ii) claims benefit of pending prior U.S. Provisional Patent Application Ser. No. 62/725,650, filed Aug. 31, 2018 by CorMedix Inc. and Bruce Reidenberg et al. for TAUROLIDINE TREATMENT FOR MYC-EXPRESSING TUMORS IN MAMMALIAN BODIES (Attorney's Docket No. CORMEDIX-35 PROV).

The three (3) above-identified patent applications are hereby incorporated herein by reference.

### FIELD OF THE INVENTION

This invention relates to therapeutic methods and compositions in general, and more particularly to therapeutic methods and compositions for the treatment of MYC-expressing tumors in mammalian bodies.

### BACKGROUND OF THE INVENTION

Taurolidine is a well known antimicrobial with a published mechanism of action and antimicrobial spectrum. Taurolidine is unstable in circulation and therefore has not been successfully developed for systemic infections. Taurolidine has demonstrated efficacy in local application for peritonitis and for prevention of infection when used as a catheter-lock solution.

Taurolidine has recently been investigated for oncolytic activity and found to have an inhibitory effect on cell lines in culture, in combination with standard chemotherapy or alone. Despite claims that in vitro inhibitory concentrations are clinically achievable, the only published human pharmacokinetic study showed NO measurable concentration of taurolidine in healthy volunteers when 5 grams of taurolidine were given intravenously by 20 minute infusion. This is believed to be due to the rapid hydrolysis of taurolidine when administered systemically in a mammalian body.

MYC oncogenes have been widely described in solid tumors and in lymphoma/leukemia.

Taurolidine has demonstrated efficacy in treating neuroblastoma in a laboratory cell line. This cell line is known to overexpress N-myc genes.

Taurolidine has demonstrated efficacy in treating ovarian cancer in a human ovarian cell tumor line implanted in mice. This cell line is known to overexpress C-myc genes.

Taurolidine has demonstrated efficacy in treating lung cancer in a laboratory cell line. This cell line is known to overexpress L-myc genes.

A need exists for a new method and composition which are effective against MYC-expressing tumors in mammalian bodies.

### SUMMARY OF THE INVENTION

In accordance with the present invention, taurolidine, and/or the hydrolysis products of taurolidine, is/are used to treat tumors that overexpress N-myc genes, C-myc genes and/or L-myc genes in mammalian bodies. Examples of tumors that may overexpress N-myc genes, C-myc genes and/or L-myc genes include, but are not limited to, lymphoma, melanoma, multiple myeloma, neuroblastoma, colon, breast and lung cancers.

The preferred hydrolysis products of taurolidine may comprise at least one from the group consisting of:
taurultam;
taurinamide;
methylene glycol;
taurultam and taurinamide in a ratio of 1 taurultam:7 taurinamide; and
taurultam, taurinamide and methylene glycol in a ratio of 1 taurultam:7 taurinamide: 1 methylene glycol.

The taurolidine is given with a dosage range of from 5 mg/kg to 280 mg/kg, with optimal range between 5 mg/kg and 60 mg/kg, from once daily through weekly, for an effective period of time based on individual patient response.

The taurultam is given with a dosage range of from 5 mg/kg to 280 mg/kg, with optimal range between 5 mg/kg and 60 mg/kg, from once daily through weekly, for an effective period of time based on individual patient response.

The taurinamide is given with a dosage range of from 5 mg/kg to 280 mg/kg, with optimal range between 5 mg/kg and 60 mg/kg, from once daily through weekly, for an effective period of time based on individual patient response.

The methylene glycol is given with a dosage range of from 2.5 mg/kg to 160 mg/kg, with optimal range between 2.5 mg/kg and 30 mg/kg, from once daily through weekly, for an effective period of time based on individual patient response.

The taurultam and taurinamide (in a ratio of 1 taurultam:7 taurinamide) is given with a dosage range of taurultam from 5 mg/kg to 280 mg/kg, with optimal range between 5 mg/kg and 40 mg/kg, combined with taurinamide with a dosage range of from 5 mg/kg to 280 mg/kg, with optimal range from 35 mg/kg to 40 mg/kg, from once daily through weekly, for an effective period of time based on individual patient response.

The taurultam, taurinamide and methylene glycol (in a ratio of 1 taurultam:7 taurinamide: 1 methylene glycol) is given with a dosage range of taurultam from 5 mg/kg to 280 mg/kg, with optimal range between 5 mg/kg and 40 mg/kg, combined with taurinamide with a dosage range of from 5 mg/kg to 280 mg/kg, with optimal range from 35 mg/kg to 40 mg/kg, further combined with methylene glycol with a dosage range of from 2.5 mg/kg to 160 mg/kg with optimal range from 5 mg/kg to 40 mg/kg, from once daily through weekly, for an effective period of time based on individual patient response.

The taurolidine, and/or the hydrolysis products of taurolidine, can be given systemically, preferably intramuscularly or intravenously.

In one preferred form of the invention, the taurolidine, and/or the hydrolysis products of taurolidine, is/are delivered systemically in a “shielded form” so that the taurolidine, or the hydrolysis products of taurolidine, can reach the site of the tumor without premature degradation, whereupon the taurolidine, or the hydrolysis products of taurolidine, can treat the tumor.

More particularly, in one preferred form of the invention, the taurolidine and/or the hydrolysis products of taurolidine can be delivered in the form of a nanoparticle, where the nanoparticle comprises a core of the taurolidine and/or the hydrolysis products of taurolidine and an exterior coating which is configured to prevent premature exposure of the taurolidine and/or the hydrolysis products of taurolidine prior to the arrival of the nanoparticle to the tumor site. The exterior coating breaks down as the nanoparticle travels from the site of the insertion to the site of the tumor so as to release the taurolidine and/or the hydrolysis products of taurolidine intact at the site of the tumor. In one preferred form of the invention, the coating comprises an absorbable polymer or lipid which breaks down as the nanoparticle travels from the site of insertion to the site of the tumor.

In another form of the invention, the taurolidine and/or the hydrolysis products of taurolidine (i.e., the active ingredient) may be delivered using a polymer system which is configured to delay premature degradation of the active ingredient. By way of example but not limitation, the taurolidine and/or the hydrolysis products of taurolidine may be “pegylated” using polyethylene glycols (PEGs) to delay premature degradation of the active ingredient.

The taurolidine, and/or the hydrolysis products of taurolidine, may be delivered as either a single agent or in combination with other oncolytic agents and/or radiotherapy.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 is a graph showing that leukemia cell lines appear more sensitive to the effects of taurolidine compared to healthy lymphocytes in vitro (not in vivo);

FIG. 2 is a graph showing that neuroblastoma cell lines are more sensitive to a decrease in viability due to taurolidine when compared to healthy fibroblasts (BJ on graph) in vitro (not in vivo);

FIGS. 3-6 are graphs or photographs showing that taurolidine given to CB57 SCID mice with measurable tumors from a neuroblastoma cell line implanted subcutaneously in the CB57 SCID mice has efficacy in IMR5 tumors and measurable efficacy in SK-N-AS tumors in vivo (not in vitro);

FIGS. 7 and 8 are graphs showing that statistically significant decreases in tumor size were achieved when taurolidine was administered to treat mice with a different cell line (SK-N-AS) also derived from neuroblastoma but overall survival was not significantly different from control;

FIG. 9 is a chart showing the effect of delayed administration of a single 3-day i.p. (intraperitoneal) bolus injection regimen of taurolidine (20 mg/mouse/injection) on the occurrence of i.p. human tumor xenografts in female nude mice after the i.p. administration of 5×10⁶ SKOV-3 human ovarian tumor cells;

FIG. 10 illustrates the mechanism for the hydrolysis of taurolidine;

FIG. 11 is a chart showing the mean pharmacokinetic parameters of taurultam; and

FIG. 12 is a chart showing the mean pharmacokinetic parameters of taurinamide.

### DETAILED DESCRIPTION OF THE INVENTION

Taurolidine was developed as an anti-infective, but has been found to have oncolytic activity against neuroblastoma tumors in a laboratory cell line. This laboratory cell line is known to overexpress N-myc genes. More particularly, taurolidine has been found to have surprising oncolytic activity in cell cultures of human cancer cells expressing N-myc, and now in a rodent cancer model based on an N-myc expressing human cancer cell line.

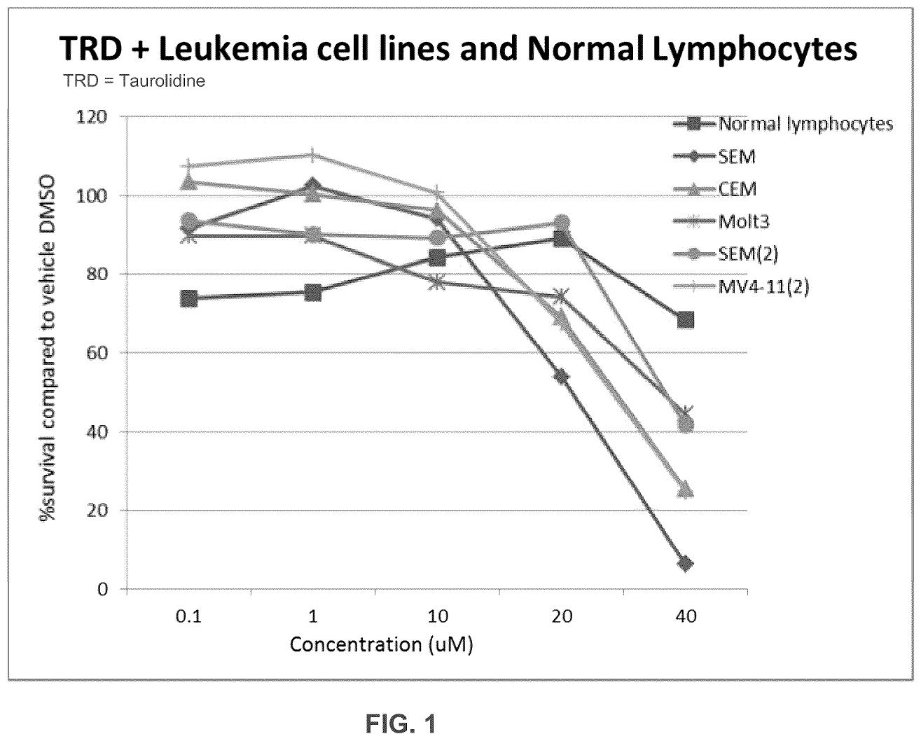

It has been found that leukemia cell lines appear more sensitive to the effects of taurolidine compared to healthy lymphocytes in vitro (not in vivo). See FIG. 1.

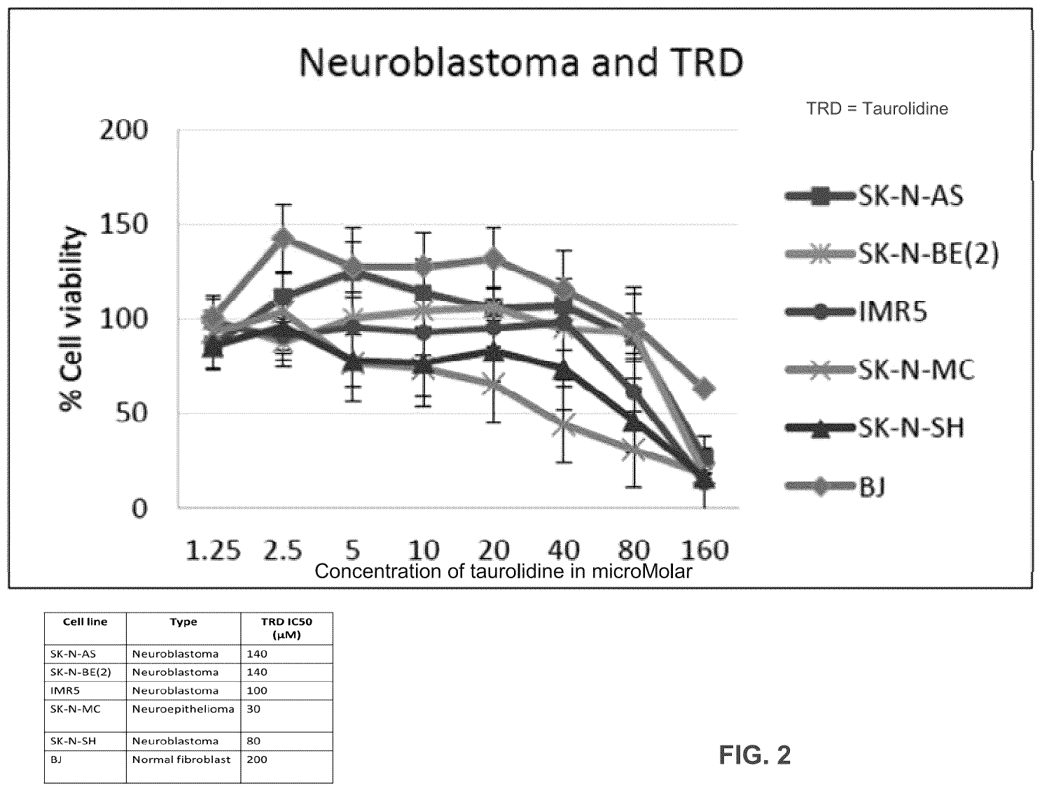

It has also been found that neuroblastoma cell lines are more sensitive to a decrease in viability due to taurolidine when compared to healthy fibroblasts in vitro (not in vivo). See FIG. 2.

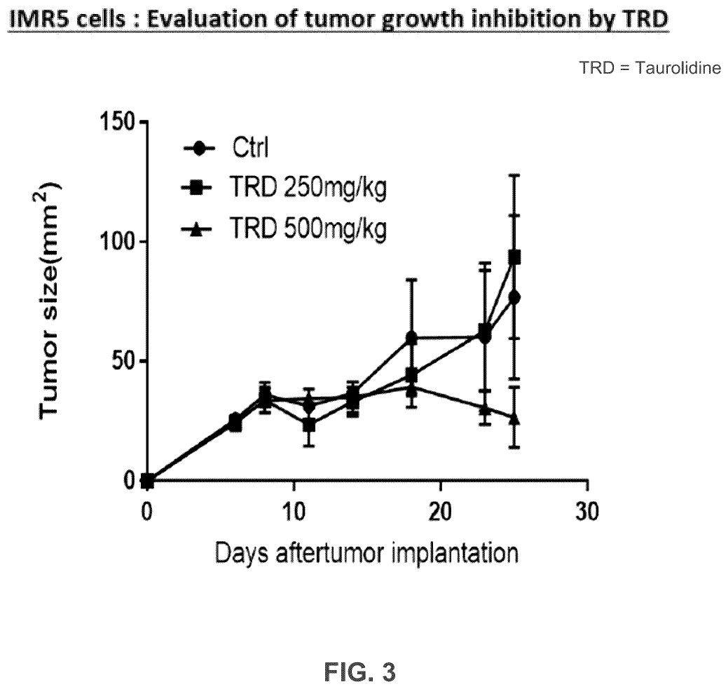

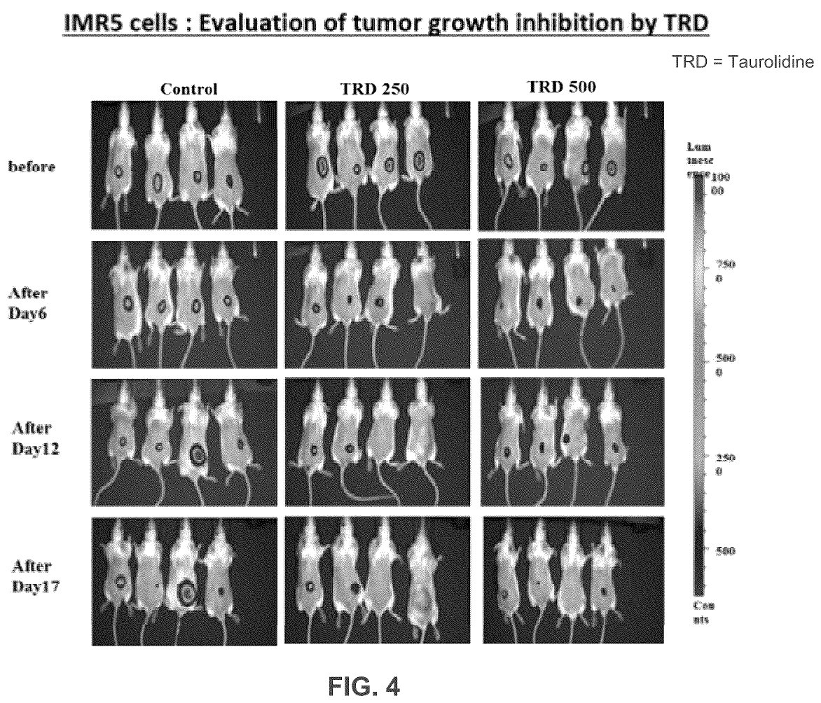

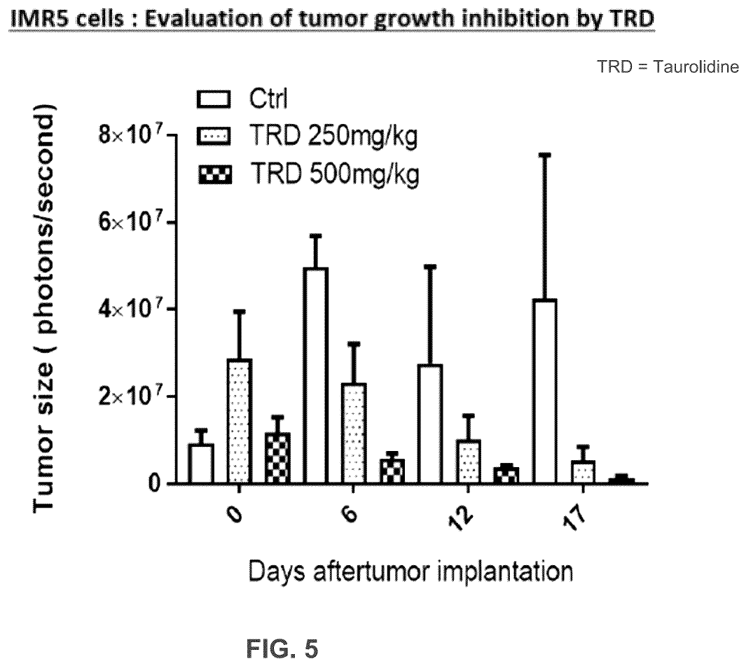

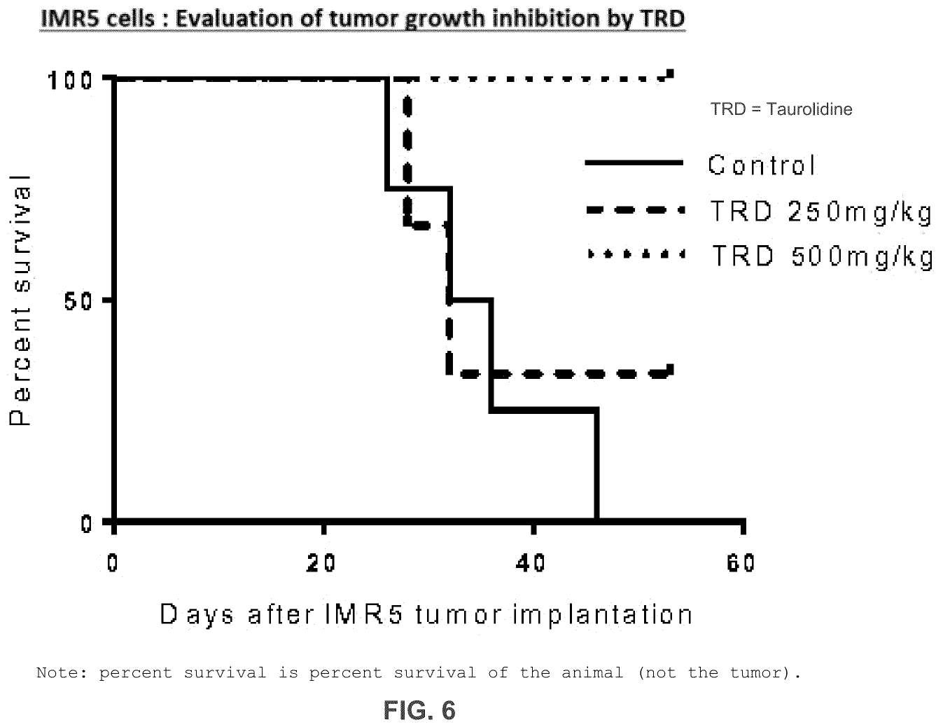

Furthermore, taurolidine given to CB57 SCID mice with measurable tumors from a neuroblastoma cell line implanted subcutaneously in the CB57 SCID mice showed dramatic efficacy in IMR5 tumors and measurable efficacy in SK-N-AS tumors in vivo (not in vitro). See FIGS. 3-6.

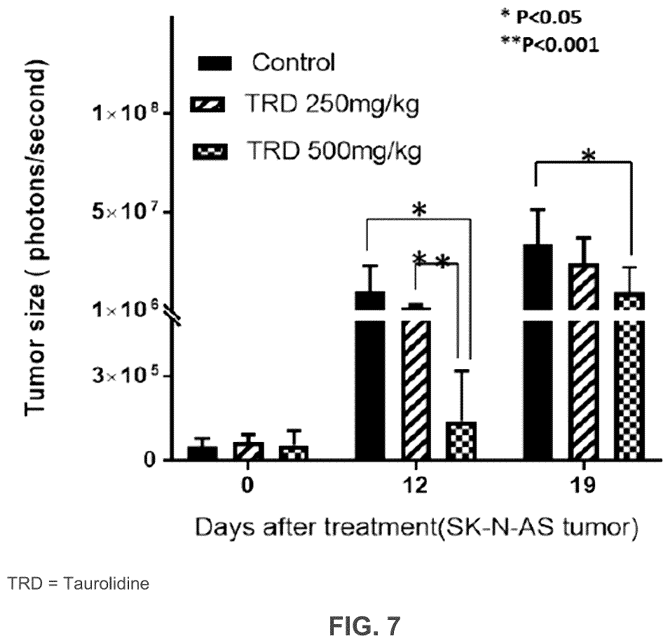

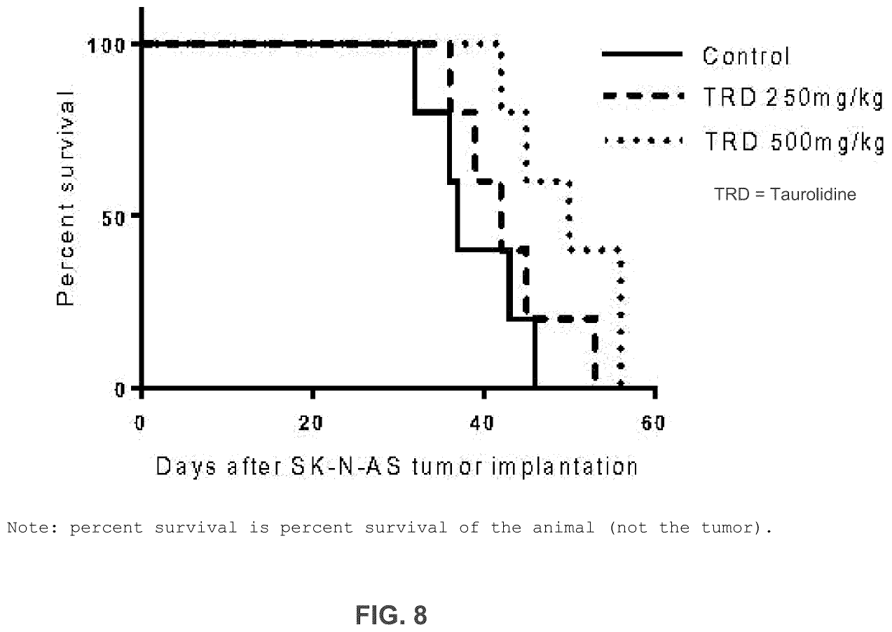

Statistically significant decreases in tumor size were achieved when taurolidine was administered to treat mice with a different cell line (SK-N-AS) also derived from neuroblastoma, though overall survival of the mice implanted with the tumor was not statistically different from the control. See FIGS. 7 and 8.

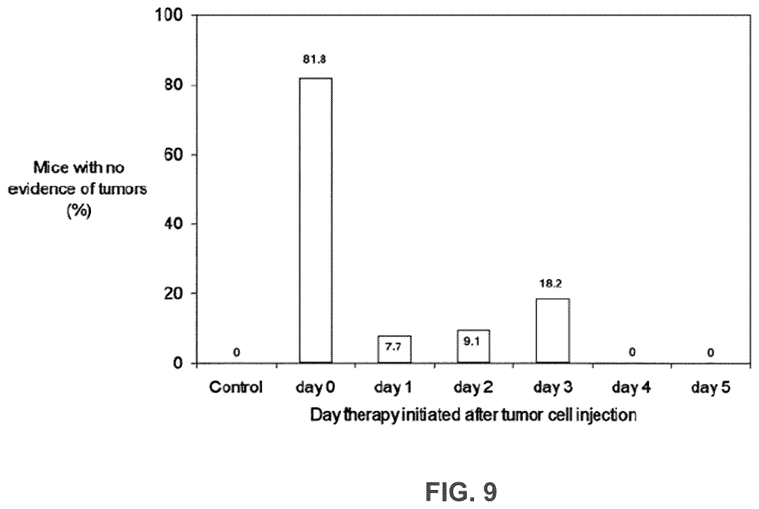

Taurolidine has also demonstrated efficacy in treating ovarian cancer in a human ovarian cell tumor line implanted in mice. This cell line is known to overexpress C-myc genes. See FIG. 9 which shows the effect of delayed administration of a single 3-day i.p. (intraperitoneal) bolus injection regimen of taurolidine (20 mg/mouse/injection) on the occurrence of i.p. human tumor xenografts in female nude mice after the i.p. administration of 5×10⁶ SKOV-3 human ovarian tumor cells. In this study, taurolidine therapy was initiated on the day of tumor cell inoculation or up to 5 days thereafter. Fourteen days after the final taurolidine injection, mice in all of the groups were sacrificed, and the peritoneal cavity was examined for the presence of tumors. Each experiment was repeated three times, and the pooled number of animals in each group ranged from 15-21 (Cancer Res., 2001 Sep. 15; 61(18):6816-21, Taurolidine: cytotoxic and mechanistic evaluation of a novel antineoplastic agent, Calabresi P1, Goulette F A, Darnowski J W).

And Taurolidine has also demonstrated efficacy in treating lung cancer in a laboratory cell line. This cell line is known to overexpress L-myc genes.

In accordance with the present invention, taurolidine, and/or the hydrolysis products of taurolidine, is/are used to treat tumors that overexpress N-myc genes, C-myc genes and/or L-myc genes in mammalian bodies. Examples of tumors that may overexpress N-myc genes, C-myc genes and/or L-myc genes include, but are not limited to, lymphoma, melanoma, multiple myeloma, neuroblastoma, colon, breast and lung cancers.

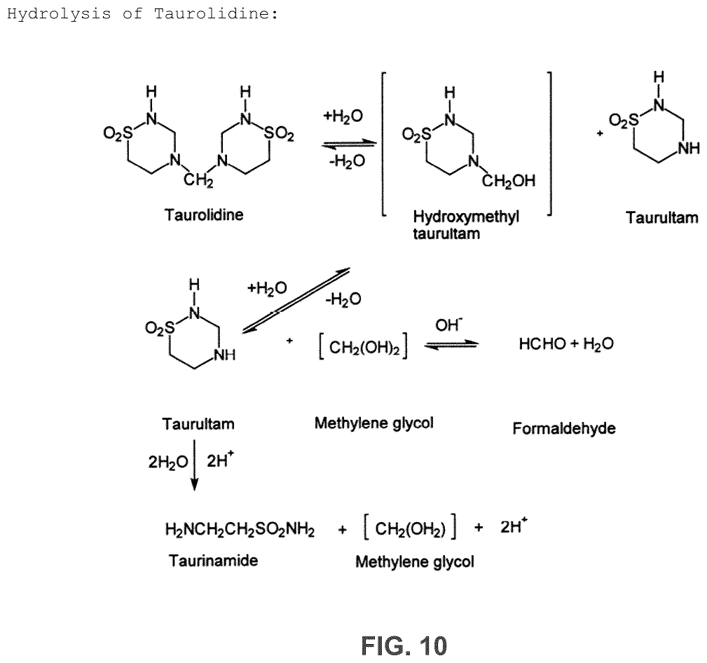

The mechanism for the hydrolysis of taurolidine is shown in FIG. 10. The preferred hydrolysis products of taurolidine that may be used to treat tumors that overexpress N-myc genes, C-myc genes and/or L-myc genes in mammalian bodies may comprise at least one from the group consisting of:
taurultam;
taurinamide;
methylene glycol;
taurultam and taurinamide in a ratio of 1 taurultam:7 taurinamide; and
taurultam, taurinamide and methylene glycol in a ratio of 1 taurultam:7 taurinamide: 1 methylene glycol.

The taurolidine is given with a dosage range of from 5 mg/kg to 280 mg/kg, with optimal range between 5 mg/kg and 60 mg/kg, from once daily through weekly, for an effective period of time based on individual patient response.

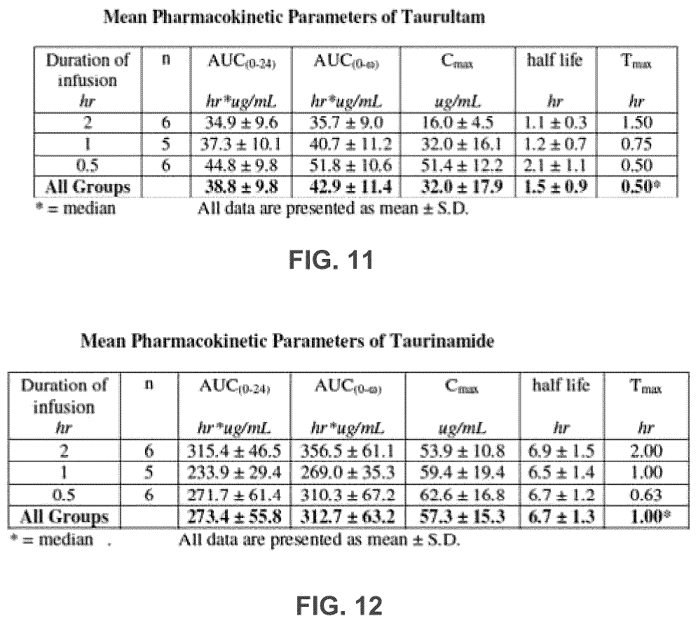

The taurultam is given with a dosage range of from 5 mg/kg to 280 mg/kg, with optimal range between 5 mg/kg and 60 mg/kg, from once daily through weekly, for an effective period of time based on individual patient response. The mean pharmacokinetic parameters of taurultam are shown in FIG. 11.

The taurinamide is given with a dosage range of from 5 mg/kg to 280 mg/kg, with optimal range between 5 mg/kg and 60 mg/kg, from once daily through weekly, for an effective period of time based on individual patient response. The mean pharmacokinetic parameters of taurinamide are shown in FIG. 12.

The methylene glycol is given with a dosage range of from 2.5 mg/kg to 160 mg/kg, with optimal range between 2.5 mg/kg and 30 mg/kg, from once daily through weekly, for an effective period of time based on individual patient response.

The taurultam and taurinamide (in a ratio of 1 taurultam:7 taurinamide) is given with a dosage range of taurultam from 5 mg/kg to 280 mg/kg, with optimal range between 5 mg/kg and 40 mg/kg, combined with taurinamide with a dosage range of from 5 mg/kg to 280 mg/kg, with optimal range from 35 mg/kg to 40 mg/kg, from once daily through weekly, for an effective period of time based on individual patient response.

The taurultam, taurinamide and methylene glycol (in a ratio of 1 taurultam:7 taurinamide: 1 methylene glycol) is given with a dosage range of taurultam from 5 mg/kg to 280 mg/kg, with optimal range between 5 mg/kg and 40 mg/kg, from once daily through weekly, combined with taurinamide with a dosage range of from 5 mg/kg to 280 mg/kg, with optimal range from 35 mg/kg to 40 mg/kg, further combined with methylene glycol with a dosage range of from 2.5 mg/kg to 160 mg/kg with optimal range from 5 mg/kg to 40 mg/kg from once daily through weekly, for an effective period of time based on individual patient response.

Dose selection for the hydrolysis products of taurolidine were calculated as follows:

$$AUC 0 - inf Taurultam / AUC 0 - inf Taurinamide = 42.9 / 312.7 = 0.14 .$$

Since the molecular weight difference is only a single methyl group, the use of weight-based AUC does not need to be corrected. Therefore the target ratio when giving taurultam and taurinamide in combination is 0.14 or 1:7. And the target ratio when giving taurultam and taurinamide and methylene glycol in combination is 1:7:1.

Effective dosage was computed by computing the human equivalent dosage from the effective mouse dose using the formula:

$$[ Human equivalent dose = mouse mg / kg dose \times 1 adult human / 12 mice \times 25 child BSA ratio / 37 adult BSA ratio = child dose in mg / kg$$

(https://www.fda.gov/downloads/drugs/guidances/ucm078932.pdf)].

The taurolidine, and/or the hydrolysis products of taurolidine, can be given systemically, preferably intramuscularly or intravenously.

In one preferred form of the invention, the taurolidine, and/or the hydrolysis products of taurolidine, is/are delivered systemically in a “shielded form” so that the taurolidine, or the hydrolysis products of taurolidine, can reach the site of the tumor without premature degradation, whereupon the taurolidine, or the hydrolysis products of taurolidine, can treat the tumor.

More particularly, in one preferred form of the invention, the taurolidine, and/or the hydrolysis products of taurolidine, is/are delivered in the form of a nanoparticle, where the nanoparticle comprises a core comprising taurolidine and/or the hydrolysis products of taurolidine, and an exterior coating which is configured to prevent premature exposure of the taurolidine, and/or the hydrolysis products of taurolidine, prior to the arrival of the nanoparticle to the tumor site. The exterior coating breaks down as the nanoparticle travels from the site of insertion to the site of the tumor so as to release the taurolidine, and/or the hydrolysis products of taurolidine, intact at the site of the tumor. In one preferred form of the invention, the coating comprises an absorbable polymer or lipid which breaks down as the nanoparticle travels from the site of insertion to the site of the tumor. By way of example but not limitation, the coating can be created from combinations of copolymers and multimers derived from polymers structured from l-lactide, glycolide, e-caprolactone, p-dioxanone, and trimethylene carbonate. The coating may also be associated with glycols such as polyethylene glycols (PEGs), which can either be linear or multi-arm structures.

If desired, the nanoparticle may comprise an excipient (e.g., a buffer for providing enhanced hydrolytic stability of the taurolidine and/or hydrolysis product within the nanoparticle).

Additionally, if desired, the nanoparticle can further comprise a coating, wherein the coating is configured to target the nanoparticle to the site of a tumor so as to improve the efficacy of the taurolidine and/or hydrolysis product for treatment of the tumor. In one preferred form of the invention, the coating comprises binding molecules which are configured to target delivery of the nanoparticle to specific tissue.

In another form of the invention, the taurolidine, and/or the hydrolysis products of taurolidine, may be delivered using a polymer system which is configured to delay premature degradation of the taurolidine, and/or the hydrolysis products of taurolidine, and/or to optimize the release properties of the taurolidine, and/or the hydrolysis products of taurolidine. By way of example but not limitation, the taurolidine, and/or the hydrolysis products of taurolidine, may be “pegylated” using polyethylene glycols (PEGs) to delay premature degradation of the taurolidine, and/or the hydrolysis products of taurolidine, and/or to optimize the release properties of the taurolidine, and/or the hydrolysis products of taurolidine.

The taurolidine (and/or the hydrolysis products of taurolidine) may be delivered as either a single agent or in combination with other oncolytic agents and/or radiotherapy. Examples of oncolytic agents that can be combined with taurolidine and/or the hydrolysis products of taurolidine for systemic delivery are platinum compounds (cisplatin, carboplatin), alkylating agents (cyclophosphamide, ifosfamide, melphalan, topoisomerase II inhibitor), vinca alkaloids (vincristine), and topoisomerase I inhibitors (topotecan and irinotecan).

### MODIFICATIONS

While the present invention has been described in terms of certain exemplary preferred embodiments, it will be readily understood and appreciated by those skilled in the art that it is not so limited, and that many additions, deletions and modifications may be made to the preferred embodiments discussed above while remaining within the scope of the present invention.

1. A method for treating a cancer which overexpresses any of N-myc genes, C-myc genes and/or L-myc genes in a mammalian body, the method comprising:
administering a composition to the mammalian body, wherein the composition comprises
taurultam and taurinamide in a weight ratio of 1 taurultam:7 taurinamide.

2.-4. (canceled)

5. The method according to claim 1 further comprising administering the composition in conjunction with an oncolytic agent and/or radiotherapy.

6.-13. (canceled)

14. The method according to claim 1 wherein the composition further comprises methylene glycol.

15.-18. (canceled)

19. The method according to claim 1 wherein the dosage range for taurultam is from 5 mg/kg to 280 mg/kg, combined with taurinamide with a dosage range of from 5 mg/kg to 280 mg/kg, from once daily through weekly, for an effective period of time based on individual patient response.

20. The method according to claim 19 wherein the dosage range for taurultam is from 5 mg/kg to 40 mg/kg, combined with taurinamide with a dosage range of from 35 mg/kg to 40 mg/kg, from once daily through weekly, for an effective period of time based on individual patient response.

21. The method according to claim 19 wherein the composition is administered in conjunction with an oncolytic agent.

22. (canceled)

23. The method according to claim 14 wherein the dosage range for taurultam is from 5 mg/kg to 280 mg/kg, combined with taurinamide with a dosage range of from 5 mg/kg to 280 mg/kg, combined with methylene glycol with a dosage range of from 2.5 mg/kg to 160 mg/kg, from once daily through weekly, for an effective period of time based on individual patient response.

24. The method according to claim 23 wherein the dosage range for taurultam is from 5 mg/kg to 40 mg/kg, combined with taurinamide with a dosage range of from 35 mg/kg to 40 mg/kg, combined with methylene glycol with a dosage range of from 5 mg/kg to 40, from once daily through weekly, for an effective period of time based on individual patient response.

25. The method according to claim 23 wherein administering the composition further comprises administering the composition in conjunction with an oncolytic agent and/or radiotherapy.

26. The method according to claim 1 wherein administering the composition comprises delivering the composition to a patient using one from the group consisting of parenteral delivery, intramuscular delivery and intravenous delivery.

27. The method according to claim 1 wherein the composition is included in a nanoparticle, and further wherein the nanoparticle is configured to delay exposure of the composition until the nanoparticle reaches the site of a tumor.

28. The method according to claim 27 wherein the nanoparticle comprises a core of the composition and an exterior coating, wherein the exterior coating is configured to prevent exposure of the composition prior to arrival of the nanoparticle at the site of the tumor.

29. The method according to claim 28 wherein the exterior coating comprises an absorbable polymer or lipid which breaks down as the nanoparticle travels from the site of insertion to the site of the tumor.

30. The method according to claim 1 wherein administering the composition comprises delivering the composition using a polymer system which is configured to delay premature degradation of the composition.

31. The method according to claim 30 further comprising pegylating the composition using polyethylene glycols (PEGs) to delay premature degradation of the composition.

32. The method according to claim 1 wherein the composition further comprises at least one from the group consisting of taurolidine, taurultam, taurinamide and methylene glycol.
